(19) United States
(12) Patent Application Publication
(10) Pub. No.: US 2025/0179411 A1
(43) Pub. Date: Jun. 5, 2025

(54) INTERACTIVE DISPLAY FOR GAS FERMENTATION SYSTEMS

(71) Applicant: LanzaTech, Inc., Skokie, IL (US)

(72) Inventors: Stacy Ann Marshall, Chicago, IL (US); Richard Charles Geoffrey Holland, Dunedin (NZ); Asela Wickum Senarath Dassanayake, Chicago, IL (US)

(73) Assignee: LanzaTech, Inc., Skokie, IL (US)

(21) Appl. No.: 18/525,642

(22) Filed: Nov. 30, 2023

(51) Int. Cl. C12M 1/36; G01N 35/00

(52) U.S. Cl. CPC C12M 41/48; C12M 21/04; G01N 35/00871; C12M 41/12; C12M 41/36; C12M 41/40; G01N 2035/0091

(57) ABSTRACT

A computing system includes a network interface circuit configured to facilitate data transmission over a network a processing circuit comprising one or more processors coupled to non-transitory memory. The processing circuit is configured to receive, from a gas fermentation system at a first sample rate, process data corresponding with measurements taken by one or more sensors located within the gas fermentation system, receive, from a sample analysis system, sample analysis data at a second sample rate, store the process data and the sample analysis data, and generate a graphical user interface including the process data received, the sample analysis data received.

### TECHNICAL FIELD

Embodiments described herein generally relate to systems and methods for gathering gas fermentation related data from multiple data sources, integrating the data, and displaying the integrated gas fermentation data. More particularly, systems and methods described herein relate to generating an interactive display based on process data from a gas fermentation system and sample analysis data from a sample analysis system.

### BACKGROUND

Gas fermentation systems may include a distributed control system (DCS) and a sample analysis system. A distributed control system (DCS) for a gas fermentation system (e.g., biogas fermentation system) is a platform configured to control one or more components of the gas fermentation system. The DCS may include a human machine interface (HMI) that enables an operator of the DCS to control the one or more components of the gas fermentation system. Further, the DCS may receive data from one or more sensors configured to record one or more data points associated with the gas fermentation system and display the data points to the operator via the HMI.

A sample analysis system may utilize sample analysis testing to determine the contents of the sample. For example, sample analysis testing may be required to determine metabolite levels within a specific reactor vessel of the gas fermentation system. Further, next-generation sequencing (NGS) may be utilized to determine contaminants present within a vessel of the gas fermentation system. However, the results of sample analysis testing may not be available in real time, as the sample analysis testing may involve time-consuming processes. Additionally, the sample analysis testing results may not be readily available to the DCS.

### SUMMARY

One embodiment of the present disclosure is related to a computing system including a network interface circuit configured to facilitate data transmission over a network and a processing circuit comprising one or more processors coupled to non-transitory memory, wherein the processing circuit is configured to receive, from a gas fermentation system at a first sample rate, process data corresponding with measurements taken by one or more sensors located within the gas fermentation system, receive, from a sample analysis system, sample analysis data at a second sample rate, store the process data and the sample analysis data, and generate a graphical user interface including the process data received, the sample analysis data received.

According to various embodiments, the process data and the sample analysis data are received over a first period of time, wherein the processing circuit is further configured to receive, from the gas fermentation system, real time process data corresponding with measurements taken by the one or more sensors located within the gas fermentation system, wherein the graphical user interface includes the process data received over the first period of time, the sample analysis data received over the first period of time, and the real time process data. The gas fermentation system may be a first gas fermentation system, wherein the processing circuit may be further configured to retrieve, from the memory, historic process data associated with a second gas fermentation system that may be separate from the first gas fermentation system retrieve, from the memory, an operator comment associated with the historic process data compare the historic process data from the second gas fermentation system with the process data from the first gas fermentation system, determine a correlation between the historic process data from the second gas fermentation system with the process data from the first gas fermentation system, and in response to determining the correlation between the historic process data from the second gas fermentation system with the process data from the first gas fermentation system, update the graphical user interface to include the operator comment.

According to various embodiments, the processing circuit may be further configured to receive, from the gas fermentation system, an operator comment during the first period of time and associate a time with the operator comment, wherein the graphical user interface further includes an indication of the operator comment and a time stamp associated with the operator comment. The processing circuit may be further configured to receive, from the sample analysis system, an operator comment during the first period of time and associate a time with the operator comment, wherein the graphical user interface further includes an indication of the operator comment and a time stamp associated with the operator comment. The sample analysis data may include at least one of a metabolite concentration, a gas composition, a broth composition, a broth physical property, proteomic data, metabolomics data, or sequencing data. The process data may include at least one of a continuous flowrate, an intermittent flow rate, a pH level, a culture growth rate, an amperage, a voltage, a pressure, a temperature, or a rotational speed of a motor. Determining the correlation between the historic process data from the second gas fermentation system with the process data from the first gas fermentation system may include calculating one or more performance indicators of the first gas fermentation system based on at least one of the received process data or the received analysis data and determining the one or more performance indicators corresponds with one or more historic performance indicators of the second gas fermentation system. The one or more performance indicators may include at least one of a substrate utilization, a productivity level, or a concentration of microbial biomass level. The gas fermentation system and the sample analysis system may be implemented on a shared computing system.

Another embodiment of the present disclosure is related to a method. The method includes receiving, from a gas fermentation system at a first sample rate, process data corresponding with measurements taken by one or more sensors located within the gas fermentation system, receiving, from a sample analysis system, sample analysis data at a second sample rate, storing, in a memory, the process data and the sample analysis data, and generating a graphical user interface including the process data received, the sample analysis data received.

The process data and the sample analysis data may be received over a first period of time. The method may further include receiving, from the gas fermentation system, real time process data corresponding with measurements taken by the one or more sensors located within the gas fermentation system, wherein the graphical user interface includes the process data received over the first period of time, the sample analysis data received over the first period of time, and the real time process data. The gas fermentation system may be a first gas fermentation system The method may further include retrieving, from the memory, historic process data associated with a second gas fermentation system that may be separate from the first gas fermentation system, retrieving, from the memory, an operator comment associated with the historic process data, comparing the historic process data from the second gas fermentation system with the process data from the first gas fermentation system, determining a correlation between the historic process data from the second gas fermentation system with the process data from the first gas fermentation system, and in response to determining the correlation between the historic process data from the second gas fermentation system with the process data from the first gas fermentation system, updating the graphical user interface to include the operator comment.

The method may further includes receiving, from the gas fermentation system, an operator comment during the first period of time and associating a time with the operator comment, wherein the graphical user interface further includes an indication of the operator comment and a time stamp associated with the operator comment. The method may include receiving, from the sample analysis system, an operator comment during the first period of time associating a time with the operator comment, wherein the graphical user interface further includes an indication of the operator comment and a time stamp associated with the operator comment. The sample analysis data may include at least one of a metabolite concentration, a gas composition, a broth composition, a broth physical property, proteomic data, metabolomics data, or sequencing data. The process data may include at least one of a continuous flowrate, an intermittent flow rate, a pH level, a culture growth rate, an amperage, a voltage, a pressure, a temperature, or a rotational speed of a motor. Determining the correlation between the historic process data from the second gas fermentation system with the process data from the first gas fermentation system may include calculating one or more performance indicators of the first gas fermentation system based on at least one of the received process data or the received analysis data and determining the one or more performance indicators corresponds with one or more historic performance indicators of the second gas fermentation system. The one or more performance indicators may include at least one of a substrate utilization, a productivity level, or a concentration of microbial biomass level. The gas fermentation system and the sample analysis system are implemented on a shared computing system.

Another embodiment of the present disclosure is related to a computing device. The computing device includes a network interface circuit configured to facilitate data transmission over a network and at least one processing circuit comprising one or more processors coupled to non-transitory memory. The at least one processing circuit is configured to receive, from a first gas fermentation system at a first sample rate, first process data corresponding with measurements taken by one or more sensors coupled to the first gas fermentation system, receive, from a first sample analysis system, first sample analysis data at a second sample rate, receive, from a second gas fermentation system at a third sample rate, second process data corresponding with measurements taken by one or more sensors coupled to the second gas fermentation system, receive, from a second sample analysis system, second sample analysis data at a fourth sample rate, normalize the first process data, the second process data, the first sample analysis data, and the second sample analysis data, receive, from a first user, a first credential defining a first access level, determine the first user does not have access to the second process data and the second sample analysis data based on the first access level, generate a first graphical user interface including the normalized first process data and the normalized first sample analysis data, and display the first graphical user interface for viewing by the first user.

According to various embodiments, the at least one processing circuit is further configured to receive, from a second user, a second credential defining a second access level, determine the second user has access to the first process data, the first sample analysis data, the second process data, and the second sample analysis data based on the second access level, generate a second graphical user interface including the normalized first process data, the normalized first sample analysis data, the normalized second process data, and the normalized second sample analysis data, and display the second graphical user interface for viewing by the first user. The first process data and the first sample analysis data may be received over a first period of time, wherein the at least one processing circuit is further configured to receive, from the first gas fermentation system, real time process data corresponding with measurements taken by the one or more sensors coupled to the first gas fermentation system, wherein the first graphical user interface includes the first process data received over the first period of time, the first sample analysis data received over the first period of time, and the real time process data. The first process data may include at least one of a continuous flowrate, an intermittent flow rate, a pH level, a culture growth rate, an amperage, a voltage, a pressure, a temperature, or a rotational speed of a motor.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements. Numerous specific details are provided to impart a thorough understanding of embodiments of the subject matter of the present disclosure. The described features of the subject matter of the present disclosure may be combined in any suitable manner in one or more embodiments and/or implementations. In this regard, one or more features of an aspect of the invention may be combined with one or more features of a different aspect of the invention. Moreover, additional features may be recognized in certain embodiments and/or implementations that may not be present in all embodiments or implementations.

### BRIEF DESCRIPTION OF THE DRAWINGS

A more complete understanding of embodiments described herein and the advantages thereof may be acquired by referring to the following description in consideration of the accompanying drawings, in which like reference numbers indicate like features, and wherein:

FIG. 1A illustrates a schematic diagram of a data processing system, according to an example embodiment;

FIG. 1B illustrates a schematic diagram of another data processing system, according to an example embodiment.

FIG. 2 illustrates a graphical user interface generated by a live dashboard circuit, according to an example embodiment;

FIG. 3 illustrates a graphical user interface generated by a performance analysis circuit, according to an example embodiment;

FIG. 4 schematically illustrates a graphical user interface generated by a fermentation management circuit, according to an example embodiment;

FIG. 5A illustrates a graphical user interface generated by an inventory management circuit, according to an example embodiment;

FIG. 5B illustrates another graphical user interface generated by an inventory management circuit, according to an example embodiment;

FIG. 6 illustrates a graphical user interface generated by a data transfer circuit, according to an example embodiment; and

FIG. 7 illustrates sample graphical user interfaces, according to an example embodiment.

FIG. 8 illustrates a method of generating a graphical user interface for display on a device of a data processing system, according to an example embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Referring to the Figures generally, systems and methods for gathering gas fermentation data from multiple data sources, aggregating the gathered data via one or more integration processes, and selectively outputting the aggregated data via one or more graphical user interfaces are disclosed according to various embodiments herein. According to various embodiments, a computing system receives gas fermentation data, including process data and sample analysis data. For example, the process data may be received from one or more sensors of a gas fermentation system and/or a distributed control system configured to control the gas fermentation system. The sample analysis data may be received from a sample analysis system configured to perform one or more analyses on one or more samples from the gas fermentation system. The process data and the sample analysis data may be integrated together to form “gas fermentation data” by, for example, normalizing the data to create a uniform file type. The gas fermentation data or parts thereof may be selectively presented via at least one a graphical user interface that may be generated and provided to a display that displays the normalized gas fermentation data to a desired recipient, such as an operator of a distributed control system.

A variety of data sources may be analyzed by the computing system when evaluating gas fermentation systems. For example, gas fermentation data may be analyzed to determine if a gas fermentation system is performing in a desired manner and further to troubleshoot any undesirable behavior in the gas fermentation system. Process data (e.g., a continuous flowrate, an intermittent flow rate, a pH level, a culture growth rate, an amperage, a voltage, a pressure, a temperature, or a rotational speed of a motor, etc.) corresponding with readings taken from one or more of various sensors in the gas fermentation system may be received and displayed to an operator of the gas fermentation system. Sample data (e.g., sample analysis data) from samples may be taken from the gas fermentation system and provided to a sample analysis system such that one or more sample analyses may be performed. The sample analysis data (e.g., metabolite concentration, a gas composition, a broth composition, a broth physical property, proteomic data, metabolomics data, or sequencing data, etc.) may further be analyzed when assessing the functionality of the gas fermentation system. However, the sample data is dissociated from the process data. For example, the process data may be recorded by sensors within the system at a first sample rate and processed in real time while the sample analysis data may take time to generate (e.g., the sample analysis data is determined at a second sample rate), and is therefore not processed in real time. Further, the sample analysis data may be generated by a different computing device and/or computing system than the process data, and therefore, may not be readily integrated with the process data. In this example, the process data and the sample analysis data may be normalized to facilitate integrating the two different types of data. Thus, the normalization may include at least one transform of a first data type into a second data type to create a uniform file type. The uniform file type may enable searching, filtering, and analytics to be performed on a combined dataset.

Process data and sample analysis data are two examples of discrete and different types of data types that may be analyzed when assessing a gas fermentation system. While both types of data may provide insight when assessing the gas fermentation system, a better understanding of the gas fermentation system's performance may be determined when considering both process data and sample analysis data collectively. As such, the systems, methods, and apparatuses described herein address at least this technical problem by receiving process data, sample analysis data, and other types of data, integrating the data via one or more normalization processes, and generating and providing an interactive graphical user interface that displays the gas fermentation data to one or more operators of, attendants of, or other users associated with the gas fermentation system. Further, in some embodiments, the systems, methods, and apparatuses may enable receiving process data and sample analysis data from multiple gas fermentation systems such that a first gas fermentation system's performance can be compared to one or more additional gas fermentation system's performance for diagnostic and/or prognostic purposes (e.g., during troubleshooting). These and other features and benefits are described more fully herein below.

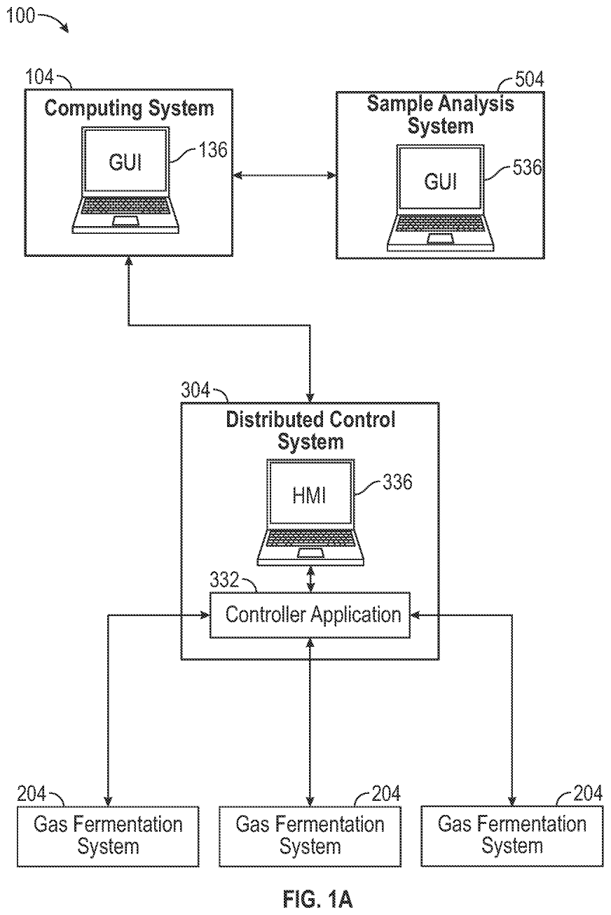

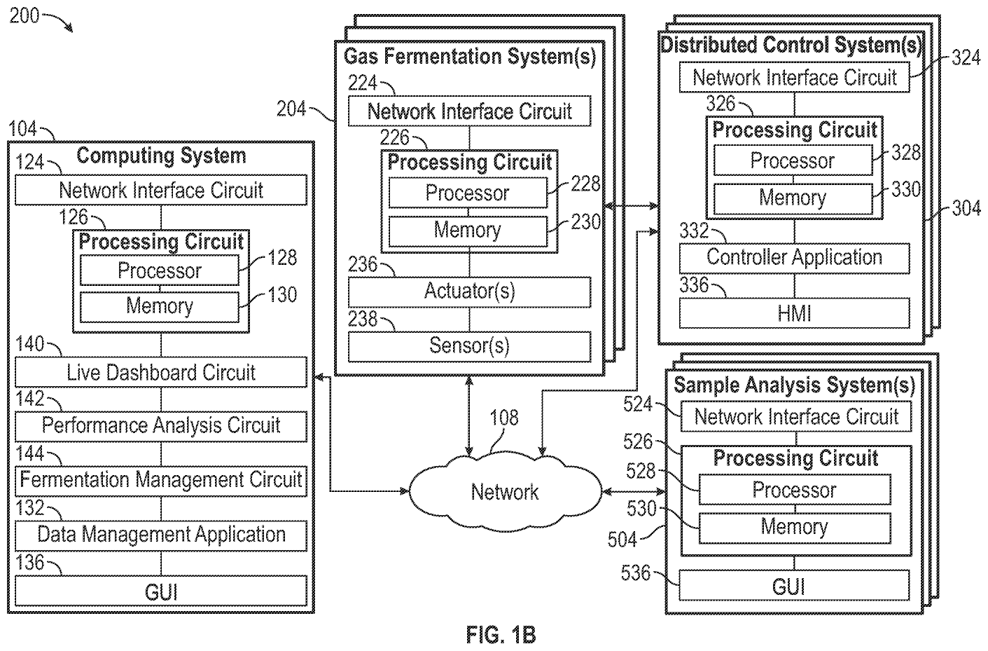

Referring now to FIG. 1A, a schematic diagram of a data processing system 100 is shown, according to an example embodiment. As is described in further detail herein, the data processing system 100 includes a computing system 104 configured to receive process data from a gas fermentation system 204 (e.g., via a distributed control system 304). The computing system 104 is further configured to receive sample analysis data from a sample analysis system 504. The computing system 104 is configured to analyze and integrate the process data and the sample analysis data, and generate and provide a graphical user interface that displays the gas fermentation data to a desired recipient, such as an operator of the distributed control system 304.

Technically, the data processing system 100 couples two or more computing systems to provide gas fermentation data in an interactive graphical user interface. The interactive graphical user interface may display real time process data, historic process data, sample analysis data, operator comments, and/or other characteristics, statistical or other analyses regarding the captured data in a single user interface. Thus, the various aspects and embodiments described herein provide a technical improvement in gas fermentation data collection and integration. The computing system 104 is configured to analyze and integrate the process data (e.g., received from the gas fermentation systems 504) and the sample analysis data (e.g., from the sample analysis system 504), and generate and provide a graphical user interface that displays the gas fermentation data to a desired recipient, such as a user of the GUI 136 included in the computing system 104 and/or an operator of the distributed control system 304.

The distributed control system (DCS) 304 includes a platform for control and operation (e.g., automated control, manual control, and/or a combination thereof) of a plant or industrial process (e.g., a gas fermentation system). The DCS 304 include a human machine interface (HMI) 336 that allows an operator to control components of the plurality of gas fermentation systems 204. For example, an operator may input commands into an input device of the HMI 336 (e.g., a keyboard, a touch screen, a mouse, etc.). The commands may be communicated to the controller application 332, which may cause one or more components of the one or more of gas fermentation systems 204 to perform the desired function.

Referring now to FIG. 1B, a schematic diagram of another data processing system 200 is shown, according to an example embodiment. The data processing system 200 may share one or more characteristics with any of the other data processing systems described herein (e.g., the data processing system 100 in FIG. 1A). For example, as shown, the data processing system 200 includes a computing system 104, a sample analysis system 504, and a plurality of gas fermentation systems 504.

As shown, the data processing system 100 includes a computing system 104, one or more gas fermentation systems 204, one or more distributed control systems 304, and one or more sample analysis systems 504. The computing system 104, the one or more gas fermentation systems 204, the one or more distributed control systems 304, and the one or more sample analysis systems 504 are shown to be communicatively and operatively coupled to each other via a network 108. The network 108 provides communicable coupling to provide and facilitate the exchange of communications (e.g., reactor related data). The network 108 may be or include one or more of a local area network, a wide area, a wired network, and/or a combination of wireless and wired networks. Examples of network configurations include the Internet, a cellular network, Wi-Fi, Wi-Max, a proprietary control systems network, etc.

As shown, the data processing system 100 includes one or more gas fermentation systems 204. Each gas fermentation system 204 may include one or more of various systems and/or devices capable of being used for a fermentation process or other chemical conversion process. For example, the gas fermentation system may include one or more reactor systems having one or more vessels or containers in which one or more gas and liquid streams or flows may be introduced for bubble generation and/or fine bubble generation, and for subsequent gas-liquid contacting, gas-absorption, biological or chemical reaction (e.g., microbial fermentation).

The term “microbial fermentation” or “fermentation” or “gas fermentation” and the like may be interpreted as the process which receives one or more substrate, such as syngas produced by gasification, and produces one or more product through the utilization of one or more C1-fixing microorganisms. A “C1-fixing microorganism” may be a microorganism that has the ability to produce one or more products from a C1-carbon source. Typically, microorganisms disclosed herein may be a C1-fixing bacterium. “C1” may refer to a one-carbon molecule, for example, CO, CO₂, CH₄, or CH₃OH. “C1-oxygenate” may refer to a one-carbon molecule that also includes at least one oxygen atom, for example, CO, CO₂, or CH₃OH. “C1-carbon source” may refer to a one carbon-molecule that serves as a partial or sole carbon source for the microorganism of the invention. For example, a C1-carbon source may include one or more of CO, CO₂, CH₄, CH₃OH, or CH₂O₂. In some examples, the C1-carbon source may include one or both of CO and CO₂. The fermentation process may include the use of one or more reactors (e.g., bioreactors). The phrases “fermenting,” “fermentation process” or “fermentation reaction” and the like, as used herein, are intended to encompass both the growth phase and product biosynthesis phase of the gaseous substrate. Examples of C1-fixing microorganisms may include Moorella, Clostridium, Ruminococcus, Acetobacterium, Eubacterium, Butyribacterium, Oxobacter, Methanosarcina, Desulfotomaculum, Clostridium autoethanogenum, and combinations thereof.

Each gas fermentation system 204 may include any type of computing device that may be used to access, research, develop, and/or modify reactor related data. Further, the computing device may be used to implement or modify one or more control processes (e.g., cause a valve position to change to affect flow rates, cause heating elements to increase or decrease output, open or close one or more vents, etc.). As shown, the gas fermentation system 204 includes a network interface circuit 224 configured to enable the gas fermentation system 204 to exchange information over a network 108, a processing circuit 226, and a Human Machine Interface (HMI) 236. The network interface circuit 224 can include program logic that facilitates connection of the gas fermentation system 204 to the network 108. The network interface circuit 224 supports wired and/or wireless communications between the gas fermentation system 204 and other systems, such as the distributed control system 304 and the computing system 104. For example, the network interface circuit 224 can include a cellular modem, a Bluetooth transceiver, a radio-frequency identification (RFID) transceiver, and a near-field communication (NFC) transmitter. In some embodiments, the network interface circuit 224 includes the hardware and machine-readable media sufficient to support communication over multiple channels of data communication. Further, in some embodiments, the network interface circuit 224 includes cryptography capabilities to establish a secure or relatively secure communication session between the gas fermentation system 204 and the computing system. In this regard, reactor related data may be encrypted and transmitted to prevent or substantially prevent a threat of hacking.

The at least one processing circuit 226 is shown to include at least one processor 228 and at least one memory 230. Each of the at least one processor 228 may be implemented as one or more application specific integrated circuits (ASICs), field programmable gate arrays (FPGAs), a group of processing components, or other suitable electronic processing components. Each of the at least one memory 230 may be one or more devices (e.g., RAM, ROM, Flash memory, hard disk storage) for storing data (e.g., reactor related data) and/or computer code for completing and/or facilitating the various processes described herein. The memory 230 may be or include non-transient volatile memory, non-volatile memory, and non-transitory computer storage media. The memory 230 may include database components, object code components, script components, or any other type of information structure for supporting the various activities and information structures described herein. The memory 230 may be communicably coupled to the processor 228 and include computer code or instructions for executing one or more processes described herein.

As shown, the gas fermentation system 204 includes one or more sensors 238. The sensors 238 may be configured to measure process data associated with the gas fermentation system 204 and communicate the process data with the processing circuit 226 such that the network interface circuit 224 can provide the process data to the computing system 104 and/or the distributed control system 304. The sensors 238 may include any type of sensor 238 included in a gas fermentation system 204, such as, but not limited to, pressure sensors configured to provide pressure data, temperature sensors configured to provide temperature data, flow rate sensors configured to provide flow rate data, humidity sensors configured to provide humidity data, etc.

The one or more sensors 238 may include real sensors and/or virtual (i.e., a non-physical sensor that is structured as program logic in the controller application 332 that makes various estimations or determinations). For example, a flow rate sensor may be a real or virtual sensor arranged to measure or otherwise acquire data, values, or information indicative of a flow rate through a conduit in a gas fermentation system (typically expressed in volume-per-time). The sensor is coupled to the engine (when structured as a real sensor), and is structured to send a signal to the controller application 332 indicative of the flow rate. When structured as a virtual sensor, at least one input may be used by the controller application 332 in an algorithm, model, lookup table, etc. to determine or estimate a parameter of the conduit (e.g., pressure). Any of the sensors 238 described herein may be real or virtual.

As shown, the data processing system 100 includes one or more distributed control systems (DCS) 304. The distributed control system(s) (DCS) 304 include a platform for control and operation (e.g., automated control, manual control, and/or a combination thereof) of a plant or industrial process (e.g., a gas fermentation system). As discussed further herein, each DCS 304 may include a human machine interface (HMI) that allows an operator to control components of the DCS 304. For example, each distributed control system 304 may include any device capable of controlling one or more components of a gas fermentation system 204. The distributed control system 304 may be configured to cause one or more automated processes at the gas fermentation system 204 to occur. According to various embodiments, the distributed control system 304 and the gas fermentation system 204 are proximate one another. However, according to other embodiments, the distributed control system 304 remotely controls the gas fermentation system 204.

Each distributed control system 304 may include any type of computing device (e.g., a laptop, a desktop computer, a tablet, etc.) that may be used to access, research, develop, and/or modify reactor related data or control one or more components of a gas fermentation system 204. As shown, the distributed control system 304 includes a network interface circuit 324 configured to enable the distributed control system 304 to exchange information over the network 108, a processing circuit 326, and a Human Machine Interface (HMI) 336. The network interface circuit 324 can include program logic that facilitates connection of the distributed control system 304 to the network 108. The network interface circuit 324 supports communications between the distributed control system 304 and other systems, such as the gas fermentation system 204 and the computing system 104. It should be appreciated that the gas fermentation system 204 and the distributed control system 304 may be in direct communication with one another. However, according to other embodiments, the gas fermentation system 204 and the distributed control system 304 may not be in direct communication with each other. For example, information and/or controls may be communicated between the gas fermentation system 204 and the distributed control system 304 via the network 108.

According to various embodiments, the network interface circuit 324 can include a cellular modem, a Bluetooth transceiver, a radio-frequency identification (RFID) transceiver, and a near-field communication (NFC) transmitter. In some embodiments, the network interface circuit 324 includes the hardware and machine-readable media sufficient to support communication over multiple channels of data communication. Further, in some embodiments, the network interface circuit 324 includes cryptography capabilities to establish a secure or relatively secure communication session between the distributed control system 304 and the computing system 104 or the gas fermentation system 204. In this regard, reactor related data may be encrypted and transmitted to prevent or substantially prevent a threat of hacking.

The processing circuit 326 is shown to include a processor 328 and a memory 330. The processor 328 may be implemented as one or more application specific integrated circuits (ASICs), field programmable gate arrays (FPGAs), a group of processing components, or other suitable electronic processing components. The memory 330 may be one or more devices (e.g., RAM, ROM, Flash memory, hard disk storage) for storing data (e.g., reactor related data) and/or computer code for completing and/or facilitating the various processes described herein. The memory 330 may be or include non-transient volatile memory, non-volatile memory, and non-transitory computer storage media. The memory 330 may include database components, object code components, script components, or any other type of information structure for supporting the various activities and information structures described herein. The memory 330 may be communicably coupled to the processor 328 and include computer code or instructions for executing one or more processes described herein.

The distributed control system 304 may include a controller application 332. In the example shown, the controller application 332 may be provided and supported by the computing system 104. In some embodiments, the controller application 332 is configured to generate and provide displays for presentation/display by the distributed control system 304 (e.g., to the HMI 336 described below) that enable the operator to control one or more components of the gas fermentation system 204. Further, the controller application 332 may be configured to control, view, and/or manage gas fermentation data received from the computing system 104 and, in particular, utilize the data processing system 100. For example, the controller application 332 may receive and/or generate a graphical user interface (GUI) that enables the operator to interact with the gas fermentation system 204 as discussed further herein. Accordingly, the controller application 332 is configured to send information (e.g., reactor related data) to, and receive information from, the computing system 104.

The controller application 332 is shown as an application on the distributed control system 304. The controller application 332 may be downloaded by the distributed control system 304 prior to its usage, hard coded into the memory 130 of the distributed control system 304, or be a network-based or web-based interface application such that the distributed control system 304 may provide a web browser to access the application, which may be executed remotely from the distributed control system 304. Accordingly, the distributed control system 304 may include software and/or hardware capable of implementing a network-based or web-based application. For example, in some instances, the controller application 332 includes software such as HTML, XML, WML, SGML, PHP (Hypertext Preprocessor), CGI, and like languages.

In some embodiments, the operator interacts with the controller application 332 via an HMI 336. The HMI 336 can include hardware and/or associated logics that enable the operator at the distributed control system 304 to exchange information with the distributed control system 304. For example, the HMI 336 may enable an operator to add operator comments that are provided to the computing system 104 along with other gas fermentation data. An input component of the HMI 336 can allow the operator to provide information to the distributed control system 304. The input component may include various hardware and/or associated logics such as, for example, a mechanical keyboard, a mechanical mouse, a touchscreen, a microphone, a camera, a fingerprint scanner, etc. Likewise, an output component of HMI 336 can include hardware and associated logics that allow the distributed control system 304 to provide information to the operator. For example, the output component may include a digital or touchscreen display, a speaker, illuminating icons, LEDs, etc. In this way, the operator can interact with the controller application 332. For example, the operator may provide login information (e.g., operator name, password, etc.) by typing on a mechanical keyboard or touchscreen keyboard included in the HMI 336 and be provided account information on a digital display component of the HMI 336.

As shown, the data processing system 100 includes one or more sample analysis systems 504. The sample analysis systems 504 may include one or more testing stations configured to determine analytical data related to the gas fermentation process. For example, an operator of the sample analysis systems 504 may perform one or more test on a sample taken from the gas fermentation system 204 to determine one or more sample data points.

Each of the sample analysis systems 504 are configured to determine or otherwise receive (e.g., via a manual input into an GUI 536) sample analysis data. For example, a sample may be taken from a reactor of the gas fermentation system 204 and sample analyses may be subsequently performed on the sample to determine sample analysis data (e.g., a metabolite concentration, a gas composition, a broth composition, a broth physical property, proteomic data, metabolomics data, or sequencing data.) The sample analysis data may then be provided to the computing system 104 for further analysis and subsequent processing, as described further below.

As shown, one or more sample analysis systems 504 includes a network interface circuit 524 configured to enable the one or more sample analysis systems 504 to exchange information over the network 108, a processing circuit 526 including a processor 538 and a memory 530, and a Human Machine Interface (HMI) 536. The network interface circuit 524 can include program logic that facilitates connection of the sample analysis system 504 to the network 108. The network interface circuit 524 supports communications between the sample analysis system 504 and other systems, such as the gas fermentation system 204 and the computing system 104. For example, the network interface circuit 524 can include a cellular modem, a Bluetooth transceiver, a radio-frequency identification (RFID) transceiver, and a near-field communication (NFC) transmitter. In some embodiments, the network interface circuit 524 includes the hardware and machine-readable media sufficient to support communication over multiple channels of data communication. Further, in some embodiments, the network interface circuit 524 includes cryptography capabilities to establish a secure or relatively secure communication session between the sample analysis system 504 and the computing system 104 or the gas fermentation system 204. In this regard, sample analysis related data may be encrypted and transmitted to prevent or substantially prevent a threat of hacking.

The at least one processing circuit 526 is shown to include at least one processor 528 and at least one memory 530. The processor 528 may be implemented as one or more application specific integrated circuits (ASICs), field programmable gate arrays (FPGAs), a group of processing components, or other suitable electronic processing components. The memory 530 may be one or more devices (e.g., RAM, ROM, Flash memory, hard disk storage) for storing data (e.g., sample analysis related data) and/or computer code for completing and/or facilitating the various processes described herein. The memory 530 may be or include non-transient volatile memory, non-volatile memory, and non-transitory computer storage media. The memory 530 may include database components, object code components, script components, or any other type of information structure for supporting the various activities and information structures described herein. The memory 530 may be communicably coupled to the processor 528 and include computer code or instructions for executing one or more processes described herein.

In some embodiments, the operator interacts with the one or more sample analysis systems 504 via an GUI 536. Like the HMI 336, the GUI 536 can include hardware and associated logics that enable the operator at the sample analysis system 504 to exchange information with the sample analysis system 504. For example, the GUI 536 may enable an operator to add operator comments that are provided to the computing system 104 along with other gas fermentation data. An input component of the GUI 536 can allow the operator to provide information to the sample analysis system 504. The input component may include various hardware and associated logics such as, for example, a mechanical keyboard, a mechanical mouse, a touchscreen, a microphone, a camera, a fingerprint scanner, etc. Likewise, an output component of GUI 536 can include hardware and associated logics that allow the sample analysis system 504 to provide information to the operator. For example, the output component may include a digital or touchscreen display, a speaker, illuminating icons, LEDs, etc. In this way, the operator can interact with the controller application 532. For example, the operator may provide login information (e.g., operator name, password, etc.) by typing on a mechanical keyboard or touchscreen keyboard included in the GUI 536 and be provided account information on a digital display component of the GUI 536.

As shown, the data processing system 100 includes a computing system 104. The computing system 104 is configured to receive gas fermentation data from multiple data sources, such as the gas fermentation system 204, the distributed control system 304, and/or the sample analysis system 504. For example, the computing system 104 may be configured to generate a graphical user interface including the process data received from the gas fermentation system 204 the distributed control system 304, and/or the sample analysis system 504 over the first period of time and the real time process data received from the gas fermentation system 204 within the graphical user interface, and provide the graphical user interface to the distributed control system 304 for display on the HMI 336. According to various embodiments, the computing system 104 may include any device capable of communicating process data to and from the distributed control system 304, receiving process data from the one or more sensors 238 of the gas fermentation system 204, and/or communicating sample analysis data to and from the sample analysis system 504. Further, according to various embodiments, the computing system 104 may be configured to control one or more sensors 238 of the gas fermentation system 204. For example, the computing system 104 may be configured to cause one or more automated processes at the gas fermentation system 204 to occur from a remote location. In this sense, according to various embodiments, one or more components of the computing system 104 may be implemented on the distributed control system 304.

The computing system 104 may include any type of computing device that may be used to access, research, develop, and/or modify reactor related data or control one or more components of a gas fermentation system 204. Further, the computing system 104 may be used to implement or modify one or more control processes (e.g., cause a valve position to change to affect flow rates, cause heating elements to increase or decrease output, open or close one or more vents, etc.). As shown, the computing system 104 includes a network interface circuit 124 configured to enable the computing system 104 to exchange information over the network 108, a processing circuit 126, and a Human Machine Interface (HMI) 136. The network interface circuit 124 can include program logic that facilitates connection of the computing system 104 to the network 108. The network interface circuit 124 supports communications between the computing system 104 and other systems, such as the gas fermentation system 204 and the computing system 104. For example, the network interface circuit 124 can include a cellular modem, a Bluetooth transceiver, a radio-frequency identification (RFID) transceiver, and a near-field communication (NFC) transmitter. In some embodiments, the network interface circuit 124 includes the hardware and machine-readable media sufficient to support communication over multiple channels of data communication. Further, in some embodiments, the network interface circuit 124 includes cryptography capabilities to establish a secure or relatively secure communication session between the computing system 104 and the computing system 104 or the gas fermentation system 204. In this regard, reactor related data may be encrypted and transmitted to prevent or substantially prevent a threat of hacking.

The processing circuit 126 is shown to include a processor 128 and a memory 130. The processor 128 may be implemented as one or more application specific integrated circuits (ASICs), field programmable gate arrays (FPGAs), a group of processing components, or other suitable electronic processing components. The memory 130 may be one or more devices (e.g., RAM, ROM, Flash memory, hard disk storage) for storing data (e.g., reactor related data) and/or computer code for completing and/or facilitating the various processes described herein. The memory 130 may be or include non-transient volatile memory, non-volatile memory, and non-transitory computer storage media. The memory 130 may include database components, object code components, script components, or any other type of information structure for supporting the various activities and information structures described herein. The memory 130 may be communicably coupled to the processor 128 and include computer code or instructions for executing one or more processes described herein.

The computing system 104 may include a data management application 132. In the example shown, the data management application 132 may be provided and supported by the computing system 104. In some embodiments, the data management application 132 is configured to generate and provide displays for presentation/display by the computing system 104 (e.g., to the GUI 136 described below) that enable the operator to control, view, and/or manage gas fermentation data received from the computing system 104 and, in particular, utilize the data processing system 100. For example, the data management application 132 may receive and/or generate a graphical user interface (GUI) that enables the operator to interact with the data management application 132 as discussed further herein. Accordingly, the data management application 132 is configured to send information (e.g., reactor related data) to, and receive information from, the computing system 104.

The data management application 132 is shown as an application on the computing system 104. The data management application 132 may be downloaded by the computing system 104 prior to its usage, hard coded into the memory 130 of the computing system 104, or be a network-based or web-based interface application such that the computing system 104 may provide a web browser to access the application, which may be executed remotely from the computing system 104. Accordingly, the computing system 104 may include software and/or hardware capable of implementing a network-based or web-based application. For example, in some instances, the data management application 132 includes software such as HTML, XML, WML, SGML, PHP (Hypertext Preprocessor), CGI, and like languages.

In some embodiments, the operator interacts with the data management application 132 via an GUI 136. The GUI 136 can include and input/output device and hardware and associated logics that enable the operator at the computing system 104 to exchange information with the computing system 104. An input component of the GUI 136 can allow the operator to provide information to the computing system 104. The input component may include various hardware and associated logics such as, for example, a mechanical keyboard, a mechanical mouse, a touchscreen, a microphone, a camera, a fingerprint scanner, etc. Likewise, an output component of GUI 136 can include hardware and associated logics that allow the computing system 104 to provide information to the operator. For example, the output component may include a digital or touchscreen display, a speaker, illuminating icons, LEDs, etc. In this way, the operator can interact with the data management application 132. For example, the operator may provide login information (e.g., operator name, password, etc.) by typing on a mechanical keyboard or touchscreen keyboard included in the GUI 136 and be provided account information on a digital display component of the GUI 136.

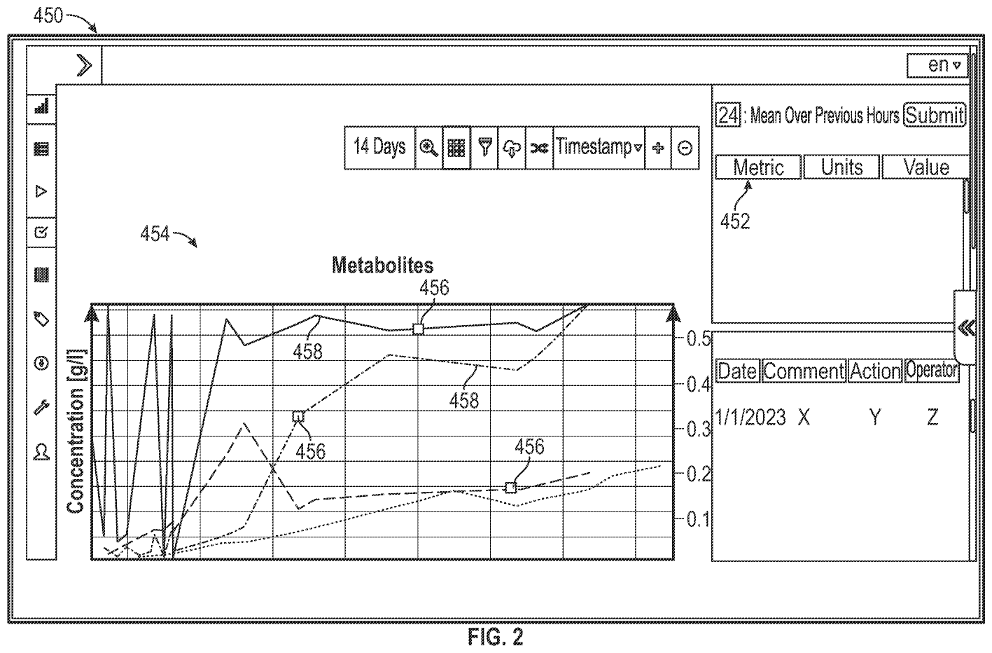

As shown, the computing system 104 includes a live dashboard circuit 140. The live dashboard circuit 140 is configured to receive gas fermentation data in real time (e.g., from the gas fermentation systems 204) and generate a graphical user interface that displays the real time data. According to various embodiments, the graphical user interface generated by the live dashboard circuit 140 may include a plurality of tabs that enables a user to switch between a plurality of gas fermentation systems 204 within the graphical user interface. The graphical user interface may be provided to the gas fermentation system 204 and/or the distributed control system 304 for display on an input/output device 236, 336. An example graphical user interface generated by the live dashboard circuit 140 is shown in FIG. 2.

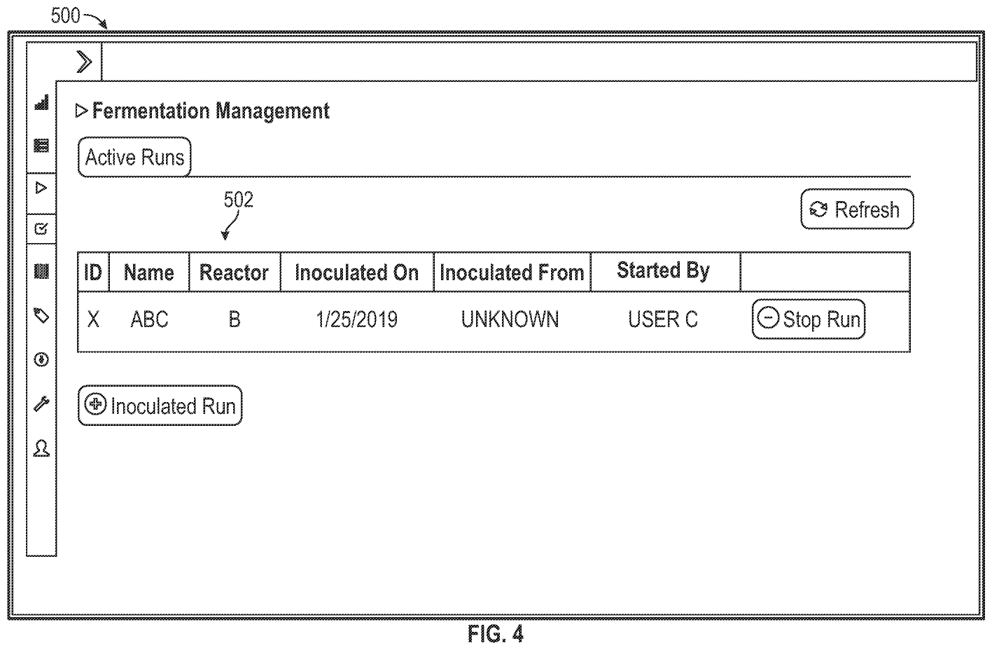

As shown, the computing system 104 includes a performance analysis circuit 142. The performance analysis circuit 142 is configured to retrieve historical gas fermentation data and generate one or more graphical user interfaces to display the historical gas fermentation data. According to various embodiments, the performance analysis circuit 142 may further be configured to receive real time gas fermentation data (e.g., from the gas fermentation systems 204) and generate a graphical user interface that displays both the historical gas fermentation data and real time gas fermentation data. In this sense, the computing system 104 integrates live gas fermentation data (e.g., process data) from the gas fermentation system 204 with stored historic data (e.g., sample analysis data) saved on the computing system 104 and generates an interactive display that includes both. An example graphical user interface generated by the performance analysis circuit 142 is shown in FIG. 4.

According to various embodiments, the performance analysis circuit 142 is further configured to determine a correlation between the historic gas fermentation data (e.g., process data, sample analysis data, etc.) and real time gas fermentation data. For example, during a real time gas fermentation run, gas fermentation data, including process data and sample analysis data are received by the performance analysis circuit 142. The performance analysis circuit 142 may analyze historic gas fermentation data and determine one or more historic gas fermentations runs included similar gas fermentation data as the real time gas fermentation run. Additionally, the performance analysis circuit 142 may determine one or more performance indicators (e.g., a substrate utilization, a productivity level, a concentration of microbial biomass level, etc.) of the real time gas fermentation run and compare the performance indicators to performance indicators from historic runs to determine if any similarities exist. In response to determining one or more historic gas fermentation runs include similar gas fermentation data as the real time gas fermentation run, the performance analysis circuit 142 may retrieve one or more operator comments included in the historic gas fermentation run data for display to an operator of the real time gas fermentation run. The historic operator comments may provide insight to the operator to facilitate troubleshooting with respect to the real time gas run.

According to various embodiments, the performance indicators are determined using a combination of process data and sample analysis data. Thus, the distributed control system 304 and the sample analysis system 504 may not be able to individually determine the performance indicators.

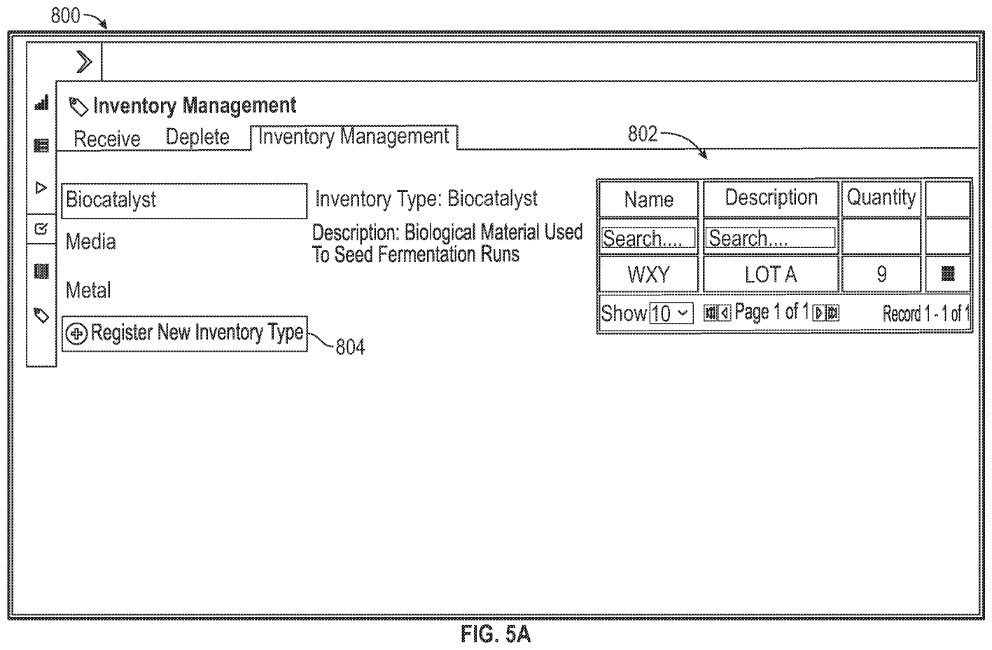

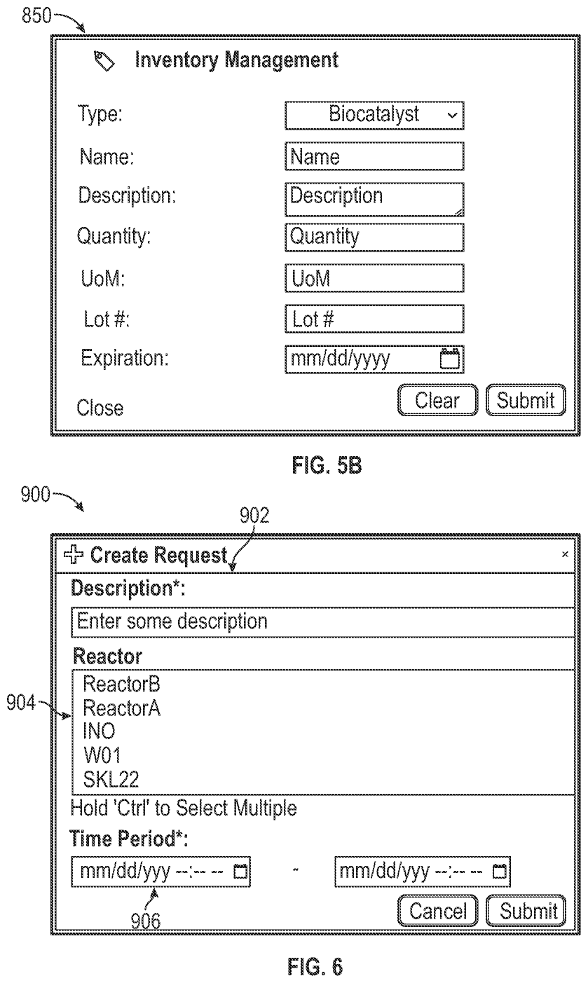

As shown, the computing system 104 includes a fermentation management circuit 144. The fermentation management circuit 144 is configured to generate a graphical user interface that enables a user to control one or more pieces of equipment of gas fermentation system 204. For example, the fermentation management circuit 144 may display one or more automated routines the gas fermentation system 204 is configured to run. By selecting one of the automated routines within the graphical user interface, the gas fermentation system 204 may run the routine as desired. The graphical user interface generated by the fermentation management circuit 144 may be displayed at the computing system 104, the gas fermentation system 204, and/or the distributed control system 304. An example graphical user interface generated by the fermentation management circuit 144 is shown in FIG. 5.

The fermentation management circuit 144 may be configured to generate a graphical user interface that enables a user to view, add, and/or modify the tags associated with one or more pieces of gas fermentation data received from the gas fermentation system 204. For example, a location of a sensor may be entered into the graphical user interface, and any data associated with that sensor may be tagged with the location. An example graphical user interface generated by the fermentation management circuit 144 is shown in FIG. 6.

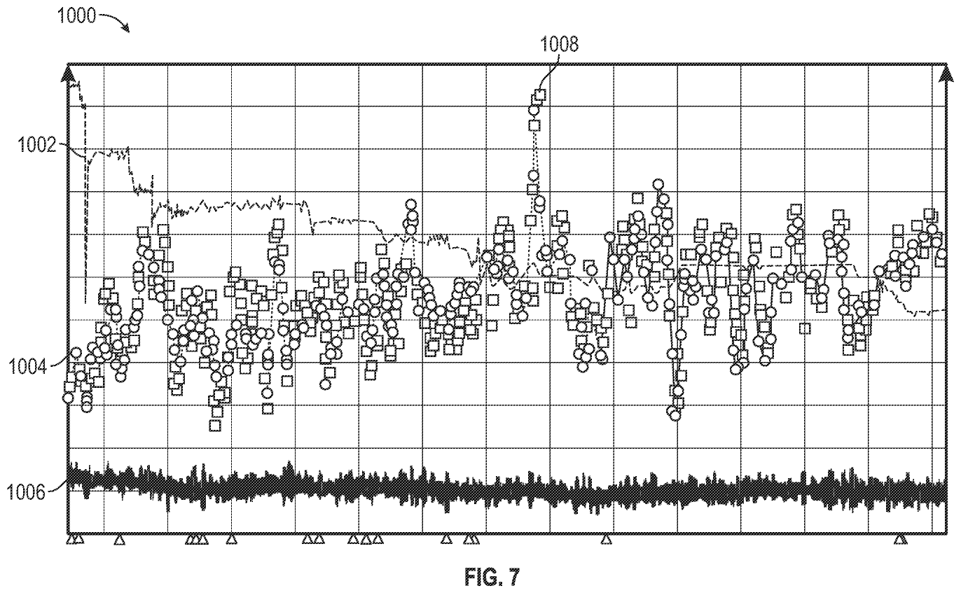

A fermentation management circuit 144 may be configured to generate a graphical user interface that enables a user view, add, and/or modify sample analysis data. For example, metabolite testing data may be added into the graphical user interface. According to various embodiments, the fermentation management circuit 144 further enables a user to generate a template for display within the graphical user interface. For example, a user may cause fermentation management circuit 144 to generate a custom template for metabolite testing, and the fermentation management circuit 144 may include the template in the graphical user interface. By utilizing the template retrieval process, a limited number of GUI templates that are specific to various data types may be utilized, thereby reducing the required computing processing to generate the GUI. An example graphical user interface generated by the fermentation management circuit 144 is shown in FIG. 7.

According to various embodiments, the fermentation management circuit 144 is configured to generate a graphical user interface that enables a user to view, add, and/or modify inventory data. For example, a user may enter in received inventory data (e.g., via the gas fermentation system 204 and/or the distributed control system 304) and the computing system 104 may track and update the inventory data as needed. For example, the fermentation management circuit 144 may track a specific chemical used in a gas fermentation system 204 and, based on a lot number associated with the chemical, track the chemical across multiple gas fermentation systems 204, which may be useful for troubleshooting. According to various embodiments, the computing system receives inventory data from gas fermentation systems 204 that are not managed by the same entity. Therefore, if a problem with a chemical from a first lot is discovered at a first gas fermentation system 204 all other gas fermentation systems 204 using that chemical from the same lot may be notified.

The fermentation management circuit 144 may be configured to generate a graphical user interface that enables a user to select specific data and export the data in a standardized format (e.g., excel, pdf, word, etc.).

Referring now to FIG. 2, a graphical user interface 450 generated by the live dashboard circuit 140 is shown, according to an example embodiment. As shown, the graphical user interface 450 displays historic and real time data (e.g., gas fermentation data 458) in a graphical plot 454. The real time data may correspond with one or more sensor measurements taken at the gas fermentation system 204 and/or one or more sample analysis data points received from the distributed control system 304. For example, the graphical user interface 450 may display gas fermentation data 458 received by the computing system 104. The graphical user interface further performance indicators 452 for over a specified period of time. Furthermore, the graphical user interface 450 displays a plurality of comments 456 corresponding with a time shown on the graphical plot 454. According to various embodiments, the plurality of comments 456 include operator comments manually entered into the data processing system 100.

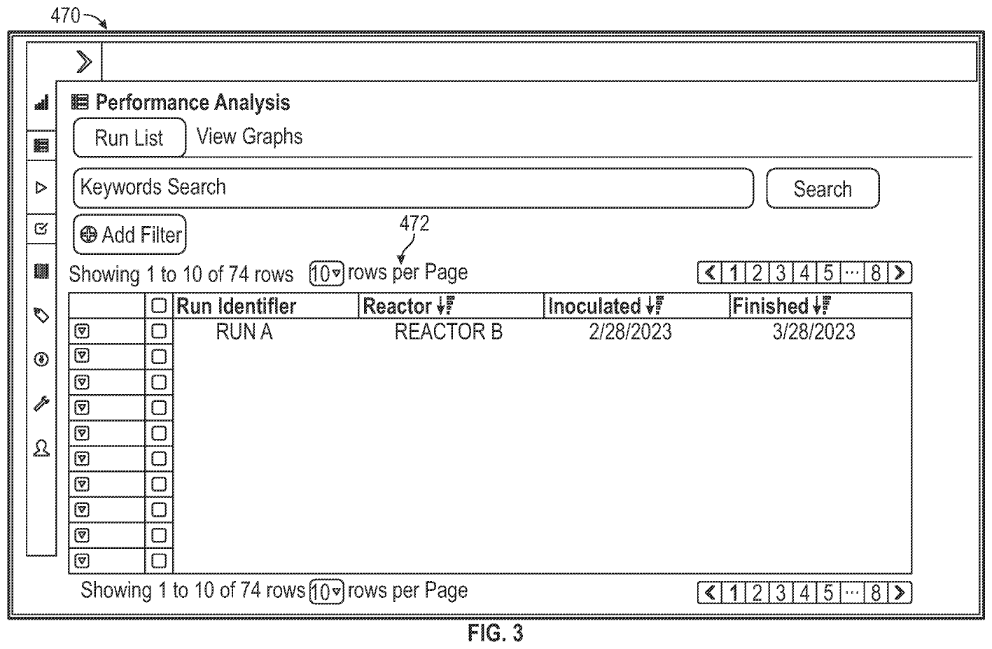

Referring now to FIG. 3, a graphical user interface 470 generated by a performance analysis circuit 142, according to an example embodiment. As shown, the graphical user interface 470 includes a list of historical runs 472 that may be selected by a user to display historical gas fermentation data and/or operator comments. Once selected, the historical data may be displayed with real time data, as is discussed further herein.

Referring now to FIG. 4, a graphical user interface 500 is shown, according to an example embodiment. The graphical user interface 500 may display a list 502 of filtered gas fermentation data associated with one or more historic gas fermentation runs. For example, the list 502 may display data associated with one or more historic gas fermentation runs, wherein one or more gas fermentation data points (e.g., process data, sample analysis data, performance indicators, etc.) is outside of a desired range. In this sense, the graphical user interface 500 may display historic gas fermentation runs that were determined to correspond with a real time gas fermentation run, which may allow a user to retrieve one or more operator comments associated with the historic gas fermentation runs to troubleshoot the real time gas fermentation run.

Referring now to FIGS. 5A and 5B, graphical user interfaces 800, 850, are shown, according to an example embodiment. The graphical user interface 800 includes a search function 802 configured to enable a user to search existing inventory items. Further the graphical user interface 800 includes an option to add a new inventory type 802. When the add a new inventory type 802 option is selected, a graphical user interface 850 may launch that enables a user to enter in inventory data. According to various embodiments, the graphical user interfaces, 800, 850 are configured to identify one or more specific lot numbers for biocatalysts used during the gas fermentation run.

Referring now to FIG. 6, a graphical user interface 900 generated by the fermentation management circuit 144 is shown, according to an example embodiment. As shown, the graphical user interface 900 enables a user to create a new data export request 902 (e.g., a batch download). The new data export request 902 is configured to export data in a standardized format. The new data export request 902 may include a gas fermentation system selection 904 and a time period selection 906 option to customize the data set that is exported.

Referring now to FIG. 7, sample graphical user interfaces 1000, 1010, 1020, 1030 for output on a user interface are shown, according to an example embodiment. For example, the performance analysis circuit 142 may generate the graphical user interfaces 1000. As shown, the graphical user interface may display real time process data, historic process data (e.g., process data 1002, 1006), sample analysis data (e.g., sample analysis data 1008, 1004), operator comments, and more in a single user interface.

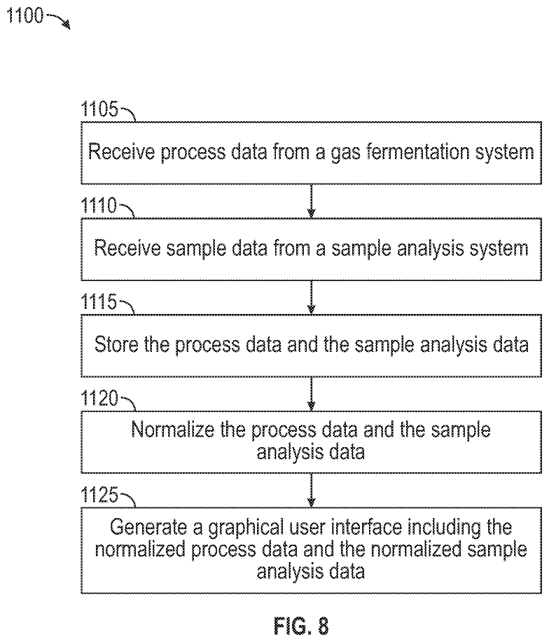

Referring now to FIG. 8, a method of generating a graphical user interface (GUI) for display on a device of a data processing system 1100 is illustrated, according to an example embodiment. As discussed above, the GUI is generated and provided to a display to present gas fermentation data (e.g., normalized process data and normalized sample data) to an operator of one or more components of a data processing system (e.g., the data processing system 100, the data processing system 200, etc.). It should be appreciated that the processes included in the method 1100 need not be performed in the order shown. Further, various processes may be omitted and additionally processes may be included in the method 1100.

Process 1105 includes receiving process data from a gas fermentation system. For example, the process data may be received by a computing system (e.g., the computing system 104) from one or more sensors of a gas fermentation system and/or a distributed control system configured to control the gas fermentation system. For example, the process data may be received from one or more sensors of a gas fermentation system and/or a distributed control system configured to control the gas fermentation system.

Process 1110 includes receiving data from a sample analysis system. The sample analysis data may be received by a computing system (e.g., the computing system 104) from a sample analysis system configured to perform analysis on one or more samples from the gas fermentation system. The sample analysis data may be received at a different sample rate (e.g., a first sample rate) than the process data (e.g., a second sample rate). The sample analysis system may be separate from the gas fermentation system. As shown in FIG. 1B, the computing system 104 is communicably coupled to both the sample analysis system 504 and the gas fermentation systems 204 (e.g., via the DCS 304) such that the computing system 104 is configured to receive both the sample analysis data and the process data.

Process 1115 includes storing the process data and the sample analysis data. For example, the process data and the sample analysis data may be stored on the memory 130 of the computing system 104. The stored process data and the stored sample analysis data may subsequently be utilized to determine a correlation between the historic gas fermentation data (e.g., process data, sample analysis data, etc.) and real time gas fermentation data. For example, during a real time gas fermentation run, gas fermentation data, including process data and sample analysis data are received. The computing system may analyze historic gas fermentation data and determine one or more historic gas fermentations runs included similar gas fermentation data as the real time gas fermentation run. Additionally, the p computing system may determine one or more performance indicators (e.g., a substrate utilization, a productivity level, a concentration of microbial biomass level, etc.) of the real time gas fermentation run and compare the performance indicators to performance indicators from historic runs to determine if any similarities exist. In response to determining one or more historic gas fermentation runs include similar gas fermentation data as the real time gas fermentation run, the computing system may retrieve one or more operator comments included in the historic gas fermentation run data for display to an operator of the real time gas fermentation run. The historic operator comments may provide insight to the operator to facilitate troubleshooting with respect to the real time gas run.

Process 1120 includes normalizing the process data and the sample analysis data. Process data and sample analysis data may be discrete and different types of data types that may be analyzed when assessing a gas fermentation system. While both types of data may provide insight when assessing the gas fermentation system, a better understanding of the gas fermentation system's performance may be determined when considering both process data and sample analysis data. The process data and the sample analysis data may be integrated together to form “gas fermentation data” by, for example, normalizing the data to create a uniform file type. It should be appreciated that, according to various embodiments, the process data and the sample analysis data may be normalized before they are stored as a part of process 1115.

Process 1125 includes generating a graphical user interface including the normalized process data and the normalized sample analysis data. For example, the computing system is configured to integrate normalized process data and the normalized sample analysis data, and generate and provide a graphical user interface that displays the gas fermentation data to a desired recipient, such as an operator of the distributed control system.

Process 1125 may also include retrieving historic normalized process data and historic normalized sample analysis data. For example, the computing system is configured to determine a correlation between the historic gas fermentation data (e.g., process data, sample analysis data, etc.) and real time gas fermentation data. For example, during a real time gas fermentation run, gas fermentation data, including process data and sample analysis data are received by the computing system. The computing system may analyze historic gas fermentation data and determine one or more historic gas fermentations runs included similar gas fermentation data as the real time gas fermentation run. Additionally, the p computing system may determine one or more performance indicators (e.g., a substrate utilization, a productivity level, a concentration of microbial biomass level, etc.) of the real time gas fermentation run and compare the performance indicators to performance indicators from historic runs to determine if any similarities exist. In response to determining one or more historic gas fermentation runs include similar gas fermentation data as the real time gas fermentation run, the p computing system may retrieve one or more operator comments included in the historic gas fermentation run data for display to an operator of the real time gas fermentation run. The historic operator comments may provide insight to the operator to facilitate troubleshooting with respect to the real time gas run.

According to various embodiments, the gas fermentation data and the historic gas fermentation data included in the GUI may depend on the credentials associated with a user requesting the GUI. For example, a user of the GUI 136 of the computing system 104 or a user of the HMI 336 of the DCS 304 may provide credentials (e.g., via the GUI 136) to the computing system 104 or the DCS 304. The credentials define a set of gas fermentation data and historic gas fermentation data that the user has access too. For example, a user (e.g., a first user) of the HIM 336 or the HMD 136 provide a first credential that only has access (e.g., a first access level) to gas fermentation data and historic gas fermentation data from the gas fermentation systems 204 associated with a specific DCS 304. For example, the user may be an operator of the DCS 304 for a company that operates a single gas fermentation system 204. In this example embodiment, the user's credentials may only allow the user to view GUIs that only include gas fermentation data and historic gas fermentation data from the single gas fermentation system 204.

According to various embodiments, separate credentials are required for each of the gas fermentation data and the historic gas fermentation data. For example, certain users may only be able to access one or the other, but not both. Additionally, specific credentials may be required to access various features within the GUIs that are configured to display gas fermentation data and/or historic gas fermentation data. For example, a first user with first user credentials may be able to access a first subset of features within the GUI and a second user with second user credentials may be able to access a second subset, different from the first subset, of features within the GUI.

In example, a second user provides a second credential that defines a second access level. The second access level may allow the user to access gas fermentation data and historic gas fermentation data associated with a plurality of gas fermentation systems 204. For example, the user may be an employee of a second company that supports a plurality of companies that each separately manage gas fermentation systems 204. The plurality of companies may not want to share their gas fermentation data and historic gas fermentation data with each other, but may be willing to share this data with the second company that supports. In this sense, the second user may compare gas fermentation data and historic gas fermentation data across various gas fermentation systems 204, which may facilitate troubleshooting individual gas fermentation systems 204 without sharing specific data between companies.

Although the present disclosure has been described in certain specific embodiments, many additional modifications and variations would be apparent to those skilled in the art. It is therefore to be understood that the present disclosure may be practiced otherwise than specifically described without departing from the scope and spirit of the present disclosure. Thus, embodiments of the present disclosure should be considered in all respects as illustrative and not restrictive. Accordingly, the scope of the disclosure should be determined not by the embodiments illustrated, but by the appended claims and their equivalents.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

1. A computing system, comprising:
a network interface circuit configured to facilitate data transmission over a network; and
at least one processing circuit comprising one or more processors coupled to non-transitory memory, wherein the at least one processing circuit is configured to:
receive, from a gas fermentation system at a first sample rate, process data corresponding with measurements taken by one or more sensors coupled to the gas fermentation system;
receive, from a sample analysis system, sample analysis data at a second sample rate;
store the process data and the sample analysis data;
normalize the process data and the sample analysis data; and
generate a graphical user interface including the normalized process data and the normalized sample analysis data, the graphical user interface generated in accordance with a template that is specific to the sample analysis data.

2. The computing system of claim 1, wherein the process data and the sample analysis data are received over a first period of time, wherein the at least one processing circuit is further configured to receive, from the gas fermentation system, real time process data corresponding with measurements taken by the one or more sensors coupled to the gas fermentation system, wherein the graphical user interface includes the process data received over the first period of time, the sample analysis data received over the first period of time, and the real time process data.

3. The computing system of claim 2, wherein the at least one processing circuit is further configured to:
receive, from the gas fermentation system, an operator comment during the first period of time; and
associate a time with the operator comment, wherein the graphical user interface further includes an indication of the operator comment and the time associated with the operator comment.

4. The computing system of claim 1, wherein the gas fermentation system is a first gas fermentation system, and wherein the at least one processing circuit is further configured to:
retrieve, from the non-transitory memory, historic process data associated with a second gas fermentation system that is separate from the first gas fermentation system;
retrieve, from the non-transitory memory, an operator comment associated with the historic process data;
compare the historic process data from the second gas fermentation system with the process data from the first gas fermentation system;
determine a correlation between the historic process data from the second gas fermentation system with the process data from the first gas fermentation system; and
update the graphical user interface to include the operator comment and indication of the determined correlation between the historic process data from the second gas fermentation system with the process data from the first gas fermentation system.

5. The computing system of claim 4, wherein determining the correlation between the historic process data from the second gas fermentation system with the process data from the first gas fermentation system includes:
determining one or more performance indicators of the first gas fermentation system based on at least one of the received process data or the received analysis data; and
determining that the one or more performance indicators corresponds with one or more historic performance indicators of the second gas fermentation system.

6. The computing system of claim 1, wherein the sample analysis data includes at least one of a metabolite concentration, a gas composition, a broth composition, a broth physical property, proteomic data, metabolomics data, or sequencing data.

7. The computing system of claim 1, wherein the process data includes at least one of a continuous flowrate, an intermittent flow rate, a pH level, a culture growth rate, an amperage, a voltage, a pressure, a temperature, or a rotational speed of a motor.

8. The computing system of claim 7, wherein the one or more performance indicators includes at least one of a substrate utilization, a productivity level, or a concentration of microbial biomass level.

9. A method, comprising:
receiving, from a gas fermentation system at a first sample rate, process data corresponding with measurements taken by one or more sensors coupled to the gas fermentation system;
receiving, from a sample analysis system that is separate from the gas fermentation system, sample analysis data at a second sample rate;
storing, in at least one memory, the process data and the sample analysis data;
normalizing the process data and the sample analysis data;
generating a graphical user interface including the normalized process data and the normalized sample analysis data, the graphical user interface generated in accordance with a template that is specific to the sample analysis data; and
selectively providing the generated graphical user interface to at least one display device in response to receiving a request for the normalized process data and the normalized sample analysis data.

10. The method of claim 9, wherein the process data and the sample analysis data are received over a first period of time, the method further comprising:
receiving, from the gas fermentation system, real time process data corresponding with measurements taken by the one or more sensors coupled to the gas fermentation system, wherein the graphical user interface includes the process data received over the first period of time, the sample analysis data received over the first period of time, and the real time process data.

11. The method of claim 10, further comprising:
receiving, from the gas fermentation system, an operator comment during the first period of time; and
associating a time with the operator comment, wherein the graphical user interface further includes an indication of the operator comment and the time associated with the operator comment.

12. The method of claim 9, wherein the gas fermentation system is a first gas fermentation system, wherein the method further comprises:
retrieving, from the at least one memory, historic process data associated with a second gas fermentation system that is separate from the first gas fermentation system;
retrieving, from the at least one memory, an operator comment associated with the historic process data;
comparing the historic process data from the second gas fermentation system with the process data from the first gas fermentation system;
determining a correlation between the historic process data from the second gas fermentation system with the process data from the first gas fermentation system; and
updating the graphical user interface to include the operator comment and an indication of the determined correlation.

13. The method of claim 12, wherein determining between the historic process data from the second gas fermentation system with the process data from the first gas fermentation system includes:
calculating one or more performance indicators of the first gas fermentation system based on at least one of the received process data or the received analysis data; and
determining the one or more performance indicators corresponds with one or more historic performance indicators of the second gas fermentation system.

14. The method of claim 12, wherein the one or more performance indicators includes at least one of a substrate utilization, a productivity level, or a concentration of microbial biomass level.

15. The method of claim 9, wherein the sample analysis data includes at least one of a metabolite concentration, a gas composition, a broth composition, a broth physical property, proteomic data, metabolomics data, or sequencing data.

16. The method of claim 9, wherein the process data includes at least one of a continuous flowrate, an intermittent flow rate, a pH level, a culture growth rate, an amperage, a voltage, a pressure, a temperature, or a rotational speed of a motor.

17. A computing device, comprising:
a network interface circuit configured to facilitate data transmission over a network; and
at least one processing circuit comprising one or more processors coupled to non-transitory memory, wherein the at least one processing circuit is configured to:
receive, from a first gas fermentation system at a first sample rate, first process data corresponding with measurements taken by one or more sensors coupled to the first gas fermentation system;
receive, from a first sample analysis system, first sample analysis data at a second sample rate;
receive, from a second gas fermentation system at a third sample rate, second process data corresponding with measurements taken by one or more sensors coupled to the second gas fermentation system;
receive, from a second sample analysis system, second sample analysis data at a fourth sample rate;
normalize the first process data, the second process data, the first sample analysis data, and the second sample analysis data;
receive, from a first user, a first credential defining a first access level;
determine the first user does not have access to the second process data and the second sample analysis data based on the first access level;
generate a first graphical user interface including the normalized first process data and the normalized first sample analysis data the first graphical user interface generated in accordance with a template that is specific to the first sample analysis data; and
display the first graphical user interface for viewing by the first user.

18. The computing device of claim 17, wherein the at least one processing circuit is further configured to:
receive, from a second user, a second credential defining a second access level;
determine the second user has access to the first process data, the first sample analysis data, the second process data, and the second sample analysis data based on the second access level;
generate a second graphical user interface including the normalized first process data, the normalized first sample analysis data, the normalized second process data, and the normalized second sample analysis data; and
display the second graphical user interface for viewing by the first user.

19. The computing device of claim 17, wherein the first process data and the first sample analysis data are received over a first period of time, wherein the at least one processing circuit is further configured to receive, from the first gas fermentation system, real time process data corresponding with measurements taken by the one or more sensors coupled to the first gas fermentation system, wherein the first graphical user interface includes the first process data received over the first period of time, the first sample analysis data received over the first period of time, and the real time process data.

20. The computing device of claim 17, wherein the first process data includes at least one of a continuous flowrate, an intermittent flow rate, a pH level, a culture growth rate, an amperage, a voltage, a pressure, a temperature, or a rotational speed of a motor.
